(19) United States
(12) Patent Application Publication
(10) Pub. No.: US 2011/0091538 A1
(43) Pub. Date: Apr. 21, 2011

(54) POLYMER ADAPTED TO RELEASE BIOACTIVE AGENTS IN VIVO, PHARMACEUTICAL COMPOSITION AND METHOD OF PREPARATION THEREOF

(75) Inventor: Benzion Geshuri, Tel Mond (IL)

(73) Assignee: Z.H.T. ENGINEERING EQUIPMENT AND TECHNOLOGIES LTD., Tel Mond (IL)

(21) Appl. No.: 12/997,978

(22) PCT Filed: Jun. 16, 2009
(86) PCT No.: PCT/IL09/00599

§ 371 Date: Jan. 6, 2011

(30) Foreign Application Priority Data
Jun. 17, 2008 (IL) 192262

(51) Int. Cl. A61K 9/48; C08F 220/10; A61K 9/14; A61K 31/78; A61K 8/81; A61P 29/00; A61P 7/04; A61P 17/02; A61P 3/02; A61P 19/00; B82Y 5/00

(52) U.S. Cl. 424/452; 526/328.5; 424/501; 424/490; 424/78.31; 424/53; 977/773; 977/810; 977/915

(57) ABSTRACT

The present invention provides a Monodisperse Polymer Particles (MPP) adapted to release alkoxy groups by means of a hydrolyser, such that Monodisperse Bioactive Polymer Particles (MBPP) are obtained in vivo. The MBPP are characterized by (a) at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule; (b) a long dimension between 0.1 and 10 micrometers; and, (c) a zeta potential value of 30 to 130 mV at pH of about 7.0. The MBPP alter, inhibit, activate, induce or otherwise affect biological or chemical events in vivo.

### BACKGROUND OF THE INVENTION

A wound may be defined as a defect, break or damage in the dermis of the skin that outcomes from physical, mechanical or thermal damage including burns. Often the wound might involve bleeding caused by damage to the dermal blood vessels. Later damage can occur due to bacterial growth or to an inflammatory response. To heal a wound, the body undertakes a series of actions collectively known as the wound healing process, which includes three acceptable phases: the inflammatory, proliferative, and remodeling phases. When a wound remains for two long in the inflammatory phase (usually longer than three months) it is considered to be a chronic wound.

In Europe and the US, the prevalence of chronic ulcerated wounds surpasses 8.5 million reported cases. The US Center for Medicare and Medicaid Services (CMS) allocates $20 billion of its yearly budget to wound care. Various products cater to these orders of magnitude, amounting to an estimated $7 billion global wound care market. With the expectancy of an aging population, which is the main group suffering from chronic wounds; these numbers are expected to grow steadily. Notwithstanding current solutions to chronic wounds, less than half of the wounds are healed. Therefore, novel and efficacious products are much needed.

The most common chronic wounds include diabetic foot ulcers, venous leg ulcers and pressure ulcers. Diabetic foot ulcers are non-healing ulcers which are one of the severe complications of diabetes patients, and the leading cause of diabetes-associated foot and leg amputations. In the US alone, approximately 3 million diabetes patients develop chronic diabetic ulcers, resulting in over 100,000 amputations a year. Three million individuals suffer from venous leg ulcers in the US alone. Ulceration is caused by the cessation or impairment of venous flow in the lower extremity. Despite of the standard compression dressing therapy, only 50-60% of the venous ulcers heal completely, and the recurrence rate is as high as 70%. Pressure ulcers develop in bedridden patients who are unable to reposition themselves to off-load weight, such as paralyzed, unconscious, or severely debilitated persons. About 23% of individuals in nursing homes facilities and 11% in acute care suffer from pressure ulcers. 86% of paraplegic patients develop a pressure ulcer.

The process of wound healing is generally considered to be divided into three overlapping phases. The first phase, which begins immediately after the first injury and breakage of the dermal and/or epidermal tissue is the inflammatory phase in which phagocyte cells ingest and destroy foreign debris and bacteria in a process termed as phagocytosis. During the phagocytosis the phagocytes release high levels of toxins that are directed to destroy the bactria and debris, but are also harmful to normal cells. The ingested bacteria and debris are gradually removed. This phase is accompanied with observable redness, heat, swelling, and pain. At the same time the proliferation phase begins whilst an array of factors are released which mobilize cells into the lesion area and promote their cell division.

The proliferative phase is characterized by angiogenesis, collagen deposition, granulation tissue formation, epithelialization, and wound contraction. In angiogenesis, new blood vessels grow from endothelial cells. In fibroplasia and granulation tissue formation, fibroplasts grow and form a new, provisional extracellular matrix (ECM) by excreting collagen and fibronectin

In epithelialization, epithelial cells crawl across the wound bed to cover it. In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis.

In the maturation and remodeling phase, collagen is remodeled and realigned along tension lines and cells that are no longer needed are removed by apoptosis.

The production of wound exudate occurs as a result of vasodilation during the early inflammatory stage of healing under the influence of inflammatory mediators such as histamine and bradykinin. It presents as serous fluid in the wound bed and is part of normal wound healing in acute wounds. However, when the wound becomes ‘chronic’ and non-healing with persistent, abnormal inflammation or when infection becomes established, exudate takes on a different guise and generates clinical challenges. In the chronic wound, exudate contains proteolytic enzymes and other components not seen in acute wounds. This type of exudate has justifiably been termed ‘a wounding agent in its own right’ because it has the capacity to degrade growth factors and peri-wound skin and predispose to inflammation. In order to develop an effective management approach, the clinician must be able to accurately assess and understand the implications of the composition and quantity of exudate present in the wound.

Wound exudate was described by the Swiss physician Paracelsus (c1491-1541) as nature's balsam. It is derived from serum through the inflammatory/extravasation process. Acute wound exudate contains molecules and cells that are vital to support the healing process. It has a high protein content (although lower than that found in serum), with a specific gravity greater than 1.020. Its composition includes electrolytes, glucose, cytokines, leukocytes, metalloproteinases, macrophages and micro-organisms. In the first 48 to 72 hours after wounding, platelets and fibrin may be present, but this reduces as bleeding diminishes. In chronic wounds the inflammatory response is altered owing to an uncontrolled expression of inflammatory mediators with a concurrent increase in vascular permeability and the amount of extravascular fluid. If the wound becomes infected, then an abrupt increase in exudate volume may be seen initially, followed by further quantitative and qualitative changes. This has been attributed in part to specific bacterial virulence mechanisms that result in vasodilation and extravasation.

Gautam et al (2001) have described a process whereby neutrophils attracted to the site of injury trigger the release of heparin-binding protein (HBP). It has also been shown that chronic leg ulcer exudate contains increased levels of HBP when compared to acute wound fluid. It is likely that HBPs are implicated in the production of increased exudate. Certain bacteria such as Pseudomonas aeruginosa stimulate the release of HBP from neutrophils, thus aggravating chronic inflammation by augmenting endothelial hyper-permeability. Recent research has indicated that some bacteria actually express histamine and thus, if present, produce an additional physiological source of histamine in the wound environment. Morganella species, for example M. morganii Gram-negative rods have been found to express histamine. Bacteria isolated from chronic wounds have been found to produce physiologically significant levels of histamine.

For these reasons it is believed that uncontrolled exudate is a key factor in the prevention of wound healing and keeping it chronic. Therefore it is crucial to manage and control exudate which is done by applying appropriate dressing, moisture maintenance, adsorption and drainage of the exudate in addition to the topical application of antimicrobial drugs. Nonetheless, a molecular approach from removal of exudate and toxins has been widely overlooked. One possible means for doing so on the molecular level is by using polymers which can absorb the harmful toxic components from the epithelial cells.

Polymers have long been coupled to drugs for a long time, as a source for controlled drug release in medical composition or as a coating for a medical implant. The drugs are either physically adsorbed in the cavities of the polymer or they are chemically bonded to it. Typically, a drug is coupled to the polymer carrier through an amid bond which is hydrolyzed enzymatically to release the drug at the site of the target, U.S. Pat. No. 5,543,391 teaches the use of peptides that may be covalently linked to the lipid membrane of the microparticle that carries them. Other chemical bonds are also utilized such as cleavage of an Si—N or Si—O bond that releases therapeutic agents from a polymer as disclosed in Patent Application 2007/0020308. Many other solutions take advantage of host-guest chemistry for delivering and releasing drugs. The most common is the use of cyclodextrin polymers as the host polymer which is demonstrated in U.S. Pat. No. 7,166,302. U.S. Pat. No. 7,160,592 teaches covalently bonding of therapeutic agents to a polyester layer that is part of a coating polymer of a medical device. U.S. Pat. No. 6,770,729 discloses a medical device coated with a polymer wherein the bioactive agent is capable of being released from the polymer coating into the environment in which the medical device is placed.

In targeted drug therapy, the polymer may also be adjunct to a targeting component such as a protein, an olygopeptide or an antigen which has a high affinity to a disordered location. U.S. Pat. No. 5,874,165 teaches the immobilization of bioactive agents unto a hydrophilic polymer which is serving as a second layer covering a support member such as a vascular graft. In their patent the inventors disclose the bounding of elements that are recognizable to the surrounding cells for biological adhesion of the graft to the tissue.

In all the above mentioned examples the polymer serves as a carrier for a therapeutic agent or a targeting component but does serve by itself as biologically active ingredient.

### SUMMARY OF THE INVENTION

It is in the scope of this invention to provide a therapeutic composition for ameliorating, relieving or healing a variety of wounds and other medical deteriorations such as burns, hemorrhage and the like, that induces granulation tissue formation, epithelialization, wound contraction, hemostasis or angiogenesis, which is able to outperform the existing products available through the prior art, in terms of a shorter healing time, a more complete recovery and ability to heal chronic wounds that the existing composition are not able to recover. The therapeutic composition comprises:
a. a component that generates charged particles with a narrow dispersion around the wounded tissue which are biologically active as a debridizer and capable of inducing granulation tissue formation, epithelialization, wound contraction, hemostasis, angiogenesis or a combination thereof.
b. a component that advances the proliferative step in the process of wound healing comprising essential ingredients for the wounded epithelial tissue,
c. optionally metal coated nano+microparticles, especially silver coated PMMA nano+microparticles,
d. an acceptable pharmaceutical carrier that carries the above two components, and optionally
e. other pharmaceutical agents.

The component that generates charged particles are preferably immiscible bio-nondegradable polymers such as polymethylmethacrylate (PMMA). These polymers comprise functional groups as part of their skeleton which upon hydrolysis of the latter generate charged polymer particles characterized by a high zeta potential and are nearly monodisperse. The in vivo generated charged particles are formulated with a culture media, comprising ingredients that allow eukaryotic cell proliferation, in an acceptable pharmaceutical carrier. The in vivo generated charged polymers are bioactive such that they promote healing of the wounded tissue. Their bioactivity may include but is not limited to functioning as a drug, especially anti-inflammatory agent, a coagulant, a hemostasis stimulant, a wound healing promoter, a proliferation stimulant, a tissue adhesive, a tissue functional stimulant, a chelator, an anti-oxidant, an inducer, an inhibitor, a labeling agent an inducer of granulation tissue formation, inducer of epithelialization, promoter of wound contraction, promoter of hemostasis and angiogenesis, and a mixture thereof.

These polymers are characterized as a naturally occurring or synthetic compound consisting of long molecular chains consisting of biologically stable backbones which are optionally crosslinked, further characterized by a molecular weight of at least 1 KD comprising between 10 to 1,000,000 repeated covalently linked small molecules with a functionality of at least one alkoxy releasing group per molecule, said polymer is further characterized by a diameter of 0.1 to 10 micrometers, and preferably in a narrow distribution around 1 micrometer.

The polymers that are in the scope of this invention are especially such that release alkoxy groups after a hydrolysis reaction of a functional group that is prone for hydrolysis such as an ester, an anhydride, an acyl halide and an amide.

The alkoxy groups are characterized by a chemical formula of RO⁻ in where R represents a radical selected from the group consisting of alkyl radicals having between 1 to 30 carbon atoms which optionally contain hetero-atoms, said alkyl radicals are potentially branched, potentially unsaturated, potentially halogenated, potentially comprise cyclic residues, potentially comprise heterocyclic residues, in which said cyclic and heterocyclic residues are at least partially fused.

It is further in the scope of this invention wherein the polymer is selected from the group consisting of polyalkylmethacrylate, melamine, carboxylated melamine, magnetic carboxylated melamine resin, polyvinylcarboxylate and polyvinylether.

It is yet further in the scope of this invention to add other pharmaceutically active agents to the composition comprising the bioactive polymer which can optionally be adsorbed to the aforementioned polymer.

It is also in the scope of this invention to further include bovine serum albumin in the composition.

It is further in the scope of this invention to include metal coated nano+microparticles in the composition.

The types of wounds which this invention may benefit are inter alia post operative wounds, burns, cuts, scratches, rheumatic disorders especially joints swelling cuts, incisions (including surgical incisions), abrasions, lacerations, fractures, contusions, and amputations epithelial wounds, especially chronic wounds in a mammal.

It is further in the scope of this invention to provide a method for healing wounds using the composition described herein.

It is still an object of the present invention to provide the BAC as defined above, especially adapted for treatments selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is still an object of the present invention to provide the MPP as defined above, especially adapted for treatments selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is one object of the present invention to provide Monodisperse Polymer Particles (MPP) adapted to release alkoxy groups by means of a hydrolyses, such that Monodisperse Bioactive Polymer Particles (MBPP) are obtained in vivo, said MBPP are characterized by
a. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
b. a long dimension between 0.1 and 10 micrometers; and
c. a zeta potential value of 30 to 130 mV at pH of about 7.0;
wherein said MBPP alter, inhibit, activate, induce or otherwise affect biological or chemical events in vivo.

It is another object of the present invention to provide the MPP as defined above, wherein the monomer of said MPP is said MBPP.

It is another object of the present invention to provide the MPP as defined above, wherein said alkoxy groups are characterized by a chemical formula of RO⁻ in which R represents a radical having between 1 to 20 carbon atoms selected from the group consisting of aryl radicals, linear alkyl radicals, branched alkyl radicals, alkyl radicals comprising heteroatoms, unsaturated alkyl radicals, halogenated alkyl radicals, alkyl radical comprising cyclic residues, alkyl radical comprising heterocyclic residues, alkyl radicals comprising at least partially fused cyclic residues, alkyl radicals comprising at least partially fused heterocyclic residues;

It is another object of the present invention to provide the MPP as defined above, wherein said hydrolyser is selected from a group consisting of a combination of a proton and a water molecule, a combination of a Louis acid and a water molecule, a hydroxide, and an ester It is another object of the present invention to provide the MPP as defined above, wherein said MPP are adapted to release alkoxy groups by means of a hydrolysis reaction, said hydrolysis reaction is selected from a group consisting of hydrolysis of an ester, hydrolysis of an acyl halide, hydrolysis of an amide, hydrolysis of an ether and hydrolysis of an anhydride.

It is another object of the present invention to provide the MPP as defined above, wherein said polymer is selected from the group consisting of a polyalkylmethacrylate with 1 to 20 carbon atoms in the carbon chain of the alkyl residue which is potentially unsaturated, potentially branched and potentially halogenated, polymethacrylate, melamine, carboxylated melamine resin with 1 to 30 carbon atoms in the carbon chain of the carboxylate residue which is potentially unsaturated, potentially branched and potentially halogenated, magnetic carboxylated melamine resin with 1 to 30 carbon atoms in the carbon chain of the carboxylate residue which is potentially unsaturated, potentially branched and potentially halogenated, silicone, polyvinylcarboxylate with 1 to 30 carbon atoms in the carbon chain of the carboxylate residue which is potentially unsaturated potentially branched and potentially halogenated, polyvinylether with 1 to 30 carbon atoms in the carbon chain of the alkyl residue which is potentially unsaturated, potentially branched and potentially halogenated, derivatives and copolymers thereof.

It is another object of the present invention to provide the MPP as defined above, wherein said polyalkylmethacrylate is polymethylmethacrylate (PMMA).

It is another object of the present invention to provide the MPP as defined above, wherein said polyalkylmethacrylate is added with ethylmethacrylate and/or methylmethacrilate such that the ratio between said of ethylmethacrylate and said methylmethacrilate ranges from about 10:90 to 90:10.

It is another object of the present invention to provide the MPP as defined above, wherein the size of said polyalkylmethacrylate ranges from about 0.5 to about 5 microns.

It is another object of the present invention to provide the MPP as defined above, wherein the viscosity of said polyalkylmethacrylate ranges from about 5 to about 28 cps units.

It is another object of the present invention to provide the MPP as defined above, with a narrow size distribution around 1 micrometer.

It is another object of the present invention to provide the MPP as defined above, wherein said alkoxy is methoxy.

It is another object of the present invention to provide the MPP as defined above, wherein said in vivo is selected from the group consisting of a cut, an incision, an abrasion, a laceration, a fracture, a contusion, a burn, an amputation, and a joint causing a rheumatic pain in a mammal.

It is another object of the present invention to provide the MPP as defined above, wherein said BP is capable of functioning as an agent from the group consisting of a drug, especially anti-inflammatory agent, a coagulant, a hemostasis stimulant, a wound healing promoter, a proliferation stimulant, a tissue functional stimulant, a chelator, an anti-oxidant, an inducer, an inhibitor, a labeling agent and a mixture thereof.

It is another object of the present invention to provide the MPP as defined above, wherein said polymer forms particles wherein the external diameter of said particles ranges from about 0.1 to about 10 micrometers.

It is another object of the present invention to provide the MPP as defined above, said polymer further consisting of adsorbed biologically active ingredients selected from the group consisting of a drug, a nutrient, a coenzyme, a vitamin, a pro-vitamin, a coagulant, a hemostasis stimulant, a tissue an anti-oxidant, an inducer, an inhibitor, a labeling agent and a mixture thereof, wherein said adsorbed biologically active ingredients a slowly released from said polymer.

It is another object of the present invention to provide a biologically active composition (BAC) comprising:
a. MPP adapted to release alkoxy groups such that MBPP characterized by
   i. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
   ii. a long dimension between 0.1 and 10 micrometers; and
   iii. a zeta potential value of 30 to 130 mV at pH of about 7.0 are obtained;
b. a culture media, comprising ingredients that allow eukaryotic cell proliferation;
c. bovine serum albumin; and
d. an acceptable topical carrier.

It is another object of the present invention to provide the BAC as defined above, wherein the monomer of said MPP is said MBPP.

It is another object of the present invention to provide the BAC as defined above, further comprising metal coated polymer nano+microparticles.

It is another object of the present invention to provide the BAC as defined above, wherein said metal is selected from the list consisting of tin, germanium, zinc, cadmium and silver.

It is another object of the present invention to provide the BAC as defined above, wherein the concentration of said polymer in said composition is between about 0.005 to about 10% (W/W).

It is another object of the present invention to provide the BAC as defined above, wherein said BAC formulated as a phase selected from the group consisting of gel, ointment, cream, liquid aerosol spray, and powder.

It is another object of the present invention to provide the BAC as defined above, wherein said BAC is embedded in a pad.

It is another object of the present invention to provide the BAC as defined above, wherein said composition is encapsulated within a biodegradable capsule.

It is another object of the present invention to provide the BAC as defined above, wherein said polymer is adapted to adsorb toxic metabolites from dead cells in the process of wound healing.

It is another object of the present invention to provide the BAC as defined above, wherein said biologically active composition is adapted to be topically applied on a wound.

It is another object of the present invention to provide the BAC as defined above, wherein said biologically active composition is adapted to be administrated by an administrating means; said administrating means selected from the group consisting of injection, oral ingestion, and oral application, nasal application, vaginal application, ear application, eye application, topical application, and anal application.

It is another object of the present invention to provide the BAC as defined above, further comprising a preservative.

It is another object of the present invention to provide the BAC as defined above, in which said preservative is selected from the group consisting of dichloro carbazole, methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol and mixtures thereof.

It is another object of the present invention to provide a method for treating a subject having a wound comprising steps of
a. obtaining a BAC comprising:
   i. MPP adapted to release alkoxy groups such that MBPP characterized by
      a. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
      b. a long dimension between 0.1 and 10 micrometers; and
      c. a zeta potential value of 30 to 130 mV at pH of about 7.0 are obtained;
   ii. a culture media, comprising ingredients that allow eukaryotic cell proliferation;
   iii. bovine serum albumin; and
   iv. an acceptable topical carrier;
b. cleaning said wound;
c. applying one to five drops/cm² of said BAC to said wound until said burn is completely wet;
d. optionally applying an effective amount of chloramphenicol:Dermagran™ 1:1 (w/w) 10 minutes after step c;
e. possibly covering said wound with a protective dressing; and
f. repeating steps (b) through (e) at least once a day for a period of at least one week, until said wound is healed.

It is another object of the present invention to provide the method as defined above, wherein cleaning of the wound is done with a saline solution.

It is another object of the present invention to provide the method as defined above, wherein said wound is a burn.

It is another object of the present invention to provide the BAC as defined above, especially adapted for treating symptoms selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is another object of the present invention to provide the MPP as defined above, especially adapted for treating symptoms selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is another object of the present invention to provide the BAC as defined above, formulated as mouthwash for oral diseases.

It is another object of the present invention to provide the MPP as defined above, formulated as mouthwash for oral diseases.

It is another object of the present invention to provide the BAC as defined above, especially adapted for treating Raynaud's Phenomenon.

It is another object of the present invention to provide the MPP as defined above, especially adapted for treating Raynaud's Phenomenon.

It is another object of the present invention to provide a Monodisperse Polymer Particles (MPP) adapted to release alkoxy groups by means of a hydrolyser, such that Monodisperse Bioactive Polymer Particles (MBPP) are obtained in vivo, said MBPP are characterized by
a. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
b. a long dimension between 0.1 and 10 micrometers; and
c. a zeta potential value of 30 to 130 mV at pH of about 7.0;
wherein said MBPP alter, inhibit, activate, induce or otherwise affect biological or chemical events in vivo.

It is another object of the present invention to provide the MPP as defined above, wherein the monomer of said MPP is said MBPP.

It is another object of the present invention to provide the MPP as defined above, wherein said alkoxy groups are characterized by a chemical formula of RO⁻ in which R represents a radical having between 1 to 20 carbon atoms selected from the group consisting of aryl radicals, linear alkyl radicals, branched alkyl radicals, alkyl radicals comprising heteroatoms, unsaturated alkyl radicals, halogenated alkyl radicals, alkyl radical comprising cyclic residues, alkyl radical comprising heterocyclic residues, alkyl radicals comprising at least partially fused cyclic residues, alkyl radicals comprising at least partially fused heterocyclic residues;

It is another object of the present invention to provide the MPP as defined above, wherein said hydrolyser is selected from a group consisting of a combination of a proton and a water molecule, a combination of a Louis acid and a water molecule, a hydroxide, and an ester It is another object of the present invention to provide the MPP as defined above, wherein said MPP are adapted to release alkoxy groups by means of a hydrolysis reaction, said hydrolysis reaction is selected from a group consisting of hydrolysis of an ester, hydrolysis of an acyl halide, hydrolysis of an amide, hydrolysis of an ether and hydrolysis of an anhydride.

It is another object of the present invention to provide the MPP as defined above, wherein said polymer is selected from the group consisting of a polyalkylmethacrylate with 1 to 20 carbon atoms in the carbon chain of the alkyl residue which is potentially unsaturated, potentially branched and potentially halogenated, polymethacrylate, melamine, carboxylated melamine resin with 1 to 30 carbon atoms in the carbon chain of the carboxylate residue which is potentially unsaturated, potentially branched and potentially halogenated, magnetic carboxylated melamine resin with 1 to 30 carbon atoms in the carbon chain of the carboxylate residue which is potentially unsaturated, potentially branched and potentially halogenated, silicone, polyvinylcarboxylate with 1 to 30 carbon atoms in the carbon chain of the carboxylate residue which is potentially unsaturated potentially branched and potentially halogenated, polyvinylether with 1 to 30 carbon atoms in the carbon chain of the alkyl residue which is potentially unsaturated, potentially branched and potentially halogenated, derivatives and copolymers thereof.

It is another object of the present invention to provide the MPP as defined above, wherein said polyalkylmethacrylate is polymethylmethacrylate (PMMA).

It is another object of the present invention to provide the MPP as defined above, wherein said polyalkylmethacrylate is added with ethylmethacrylate and/or methylmethacrilate such that the ratio between said of ethylmethacrylate and said methylmethacrilate ranges from about 10:90 to 90:10.

It is another object of the present invention to provide the MPP as defined above, wherein the size of said polyalkylmethacrylate ranges from about 0.5 to about 5 microns.

It is another object of the present invention to provide the MPP as defined above, wherein the viscosity of said polyalkylmethacrylate ranges from about 5 to about 28 cps units.

It is another object of the present invention to provide the MPP as defined above, with a narrow size distribution around 1 micrometer.

It is another object of the present invention to provide the MPP as defined above, wherein said alkoxy is methoxy.

It is another object of the present invention to provide the MPP as defined above, wherein said in vivo is selected from the group consisting of a cut, an incision, an abrasion, a laceration, a fracture, a contusion, a burn, an amputation, and a joint causing a rheumatic pain in a mammal.

It is another object of the present invention to provide the MPP as defined above, wherein said BP is capable of functioning as an agent from the group consisting of a drug, especially anti-inflammatory agent, a coagulant, a hemostasis stimulant, a wound healing promoter, a proliferation stimulant, a tissue functional stimulant, a chelator, an anti-oxidant, an inducer, an inhibitor, a labeling agent and a mixture thereof.

It is another object of the present invention to provide the MPP as defined above, wherein said polymer forms particles wherein the external diameter of said particles ranges from about 0.1 to about 10 micrometers.

It is another object of the present invention to provide the MPP as defined above, said polymer further consisting of adsorbed biologically active ingredients selected from the group consisting of a drug, a nutrient, a coenzyme, a vitamin, a pro-vitamin, a coagulant, a hemostasis stimulant, a tissue an anti-oxidant, an inducer, an inhibitor, a labeling agent and a mixture thereof, wherein said adsorbed biologically active ingredients a slowly released from said polymer.

It is another object of the present invention to provide a biologically active composition (BAC) comprising
d. MPP adapted to release alkoxy groups such that MBPP characterized by
   i. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
   ii. a long dimension between 0.1 and 10 micrometers; and
   iii. a zeta potential value of 30 to 130 mV at pH of about 7.0 are obtained;
e. a culture media, comprising ingredients that allow eukaryotic cell proliferation;
f. bovine serum albumin; and
g. an acceptable topical carrier.

It is another object of the present invention to provide the BAC as defined above, wherein the monomer of said MPP is said MBPP.

It is another object of the present invention to provide the BAC as defined above, further comprising metal coated polymer nano+microparticles.

It is another object of the present invention to provide the BAC as defined above, wherein said metal is selected from the list consisting of tin, germanium, zinc, cadmium and silver.

It is another object of the present invention to provide the BAC as defined above, wherein the concentration of said polymer in said composition is between about 0.005 to about 10% (W/W).

It is another object of the present invention to provide the BAC as defined above, wherein said BAC formulated as a phase selected from the group consisting of gel, ointment, cream, liquid aerosol spray, and powder.

It is another object of the present invention to provide the BAC as defined above, wherein said BAC is embedded in a pad.

It is another object of the present invention to provide the BAC as defined above, wherein said composition is encapsulated within a biodegradable capsule.

It is another object of the present invention to provide the BAC as defined above, wherein said polymer is adapted to adsorb toxic metabolites from dead cells in the process of wound healing.

It is another object of the present invention to provide the BAC as defined above, wherein said biologically active composition is adapted to be topically applied on a wound.

It is another object of the present invention to provide the BAC as defined above, wherein said biologically active composition is adapted to be administrated by an administrating means; said administrating means selected from the group consisting of injection, oral ingestion, and oral application, nasal application, vaginal application, ear application, eye application, topical application, and anal application.

It is another object of the present invention to provide the BAC as defined above, further comprising a preservative.

It is another object of the present invention to provide the BAC as defined above, in which said preservative is selected from the group consisting of dichloro carbazole, methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol and mixtures thereof.

It is another object of the present invention to provide a method for treating a subject having a wound. The method comprising steps selected inter alia from:
a. obtaining a BAC comprising:
   i. MPP adapted to release alkoxy groups such that MBPP characterized by
      1. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
      2. a long dimension between 0.1 and 10 micrometers; and
      3. a zeta potential value of 30 to 130 mV at pH of about 7.0 are obtained;
   ii. a culture media, comprising ingredients that allow eukaryotic cell proliferation;
   iii. bovine serum albumin; and
   iv. an acceptable topical carrier;
b. cleaning said wound;
c. applying one to five drops/cm² of said BAC to said wound until said burn is completely wet;
d. optionally applying an effective amount of chloramphenicol:Dermagrau™ 1:1 (w/w) 10 minutes after step c;
e. possibly covering said wound with a protective dressing; and
f. repeating steps (b) through (e) at least once a day for a period of at least one week, until said wound is healed.

It is another object of the present invention to provide the method as defined above, wherein cleaning of the wound is done with a saline solution.

It is another object of the present invention to provide the method as defined above, wherein said wound is a burn.

It is another object of the present invention to provide the BAC as defined above, especially adapted for treating symptoms selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is another object of the present invention to provide the MPP as defined above, especially adapted for treating symptoms selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations.

It is another object of the present invention to provide the BAC as defined above, The formulated as mouthwash for oral diseases.

It is another object of the present invention to provide the MPP as defined above, formulated as mouthwash for oral diseases.

It is another object of the present invention to provide the BAC as defined above, especially adapted for treating Raynaud's Phenomenon.

It is lastly an object of the present invention to provide the MPP as defined above, especially adapted for treating Raynaud's Phenomenon.

### BRIEF DESCRIPTION OF THE DRAWINGS

In order to understand the invention and to see how it may be implemented in practice, a preferred embodiment will now be described, by way of non-limiting example only, with reference to the accompanying drawing, in which:

FIG. 1 demonstrates the healing progress in a 78 year old female patient suffering from a pressure ulcer in the lower back that was treated by the biologically active composition.

FIG. 2 demonstrates the healing progress in a 72 year old female patient suffering from a pressure ulcer in the thigh that was treated by the biologically active composition.

FIGS. 3A-3C demonstrates the healing progress in a 62 years old male patient, administered regularly anti-coagulant drugs or blood thinning drugs.

FIGS. 4A-4D demonstrates the healing progress in a 52 years old female patient, administered regularly Coumadin and suffers from spontaneous bleeding.

FIGS. 5A-5G demonstrates the healing progress in a 50 years old female patient who suffered from Raynaud's Phenomenon.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide means and methods for a polymer adapted to release alkoxy groups in vivo such that bioactive polymer are obtained in vivo, pharmaceutical composition and method of preparation thereof.

Non-biodegradable monodisperse polymer particles capable of releasing alkoxy groups in vivo through a hydrolysis reaction while maintaining the general backbone intact are formulated in a pharmaceutical composition for the purpose of topical application on a wound. The resultant highly charged polymer particles are biologically potent thus conferring high healing potency to the topical composition which further comprises a culture media that supports tissue proliferation.

The term ‘narrow’ and ‘about’ refer herein to 10% more or less of the value which they refer to.

The term ‘monodisperse’ refers herein to a narrow distribution of sizes around a given value.

The term ‘wound’ as used herein refers to tissue damage or loss of any kind, including but not limited to, cuts, incisions (including surgical incisions), abrasions, lacerations, fractures, contusions, burns, amputations joints causing a rheumatic pain and the like.

The term ‘zeta potential’ is referred herein as the potential difference between the dispersing fluid and the stationary layer of fluid attached to the dispersed colloidal particle measured in units of mV.

The term ‘nano+microparticle’ refers herein to particles made of polymers or silica of the size of less than 1 micron.

The term “about” refers hereinafter to a range of 25% below or above the referred value.

The term “Raynaud's Phenomenon” refers hereinafter to a vasospastic disorder causing discoloration of the fingers, toes, and occasionally other extremities. This condition can also cause nails to become brittle with longitudinal ridges. The cause of the phenomenon is believed to be the result of vasospasms that decrease blood supply to the respective regions. Emotional stress and cold are classic triggers of the phenomenon, and the discoloration follows a characteristic pattern in time: white, blue and red.

Raynaud's Phenomenon comprises both Raynaud's disease (primary Raynaud's), where the phenomenon is idiopathic, and Raynaud's syndrome (secondary Raynaud's), where it is caused by some other instigating factor. Measurement of hand-temperature gradients is one tool used to distinguish between the primary and secondary forms.

The condition of Raynaud's Phenomenon causes painful, pale, cold extremities.

The polymer described hereby is characterized by Formula 1 which is defined as:

{(A-B)ₚ}_{q}{(C-B)ᵣ}ₛ

in which p, q, and r each represent an integer number equal to or greater than 1, s represents an integer number equal to or greater than 0; A and C each represent a naturally occurring or synthetic compound consisting of long molecular chains which are optionally crosslinked, are characterized by a molecular weight of at least 1 KD comprising between 10 to 1,000,000 repeated covalently linked small molecules, such that C is dissimilar to A.

A and C are covalently linked, optionally in an alternating sequence; B=VₖWₗYₘZₙ, in which k, l, m, and n each represent an integer number equal to or greater than 0, and the sum of k+l+m+n is equal or bigger than 1; V, W, Y and Z each represent a different functional group capable of producing a carboxylate anion upon hydrolysis such as CO═OR¹, —CONHR², —COX and —C═OOC, in which R¹ and R² are selected from a group consisting of a hydrogen atom, an alkyl and an aryl, and X is a halogen selected from a group consisting of fluoride, chloride and bromide}; wherein the polymer particles are stable in the carrying suspension and are capable of transforming into Monodisperse Bioactive Polymer Particles (MBPP) upon hydrolysis of at least part of said functional groups

For application to the wound or the skin, the compositions for use according to the invention may contain conventionally non-toxic pharmaceutically acceptable carriers.

The compositions for use according to the invention include all kinds of solid, semi-solid and fluid compositions. Compositions of particular relevance are e.g. pastes, ointments, hydrophilic ointments, creams, gels, hydrogels, solutions, emulsions, suspensions, lotions, liniments, shampoos, jellies, soaps, sticks, sprays, powders, films, foams, pads, sponges (e.g. collagen sponges), pads, dressings (such as, e.g., absorbent wound dressings), drenches, bandages, plasters and trans-dermal delivery systems.

The pharmaceutically acceptable excipients may include solvents, buffering agents, preservatives, humectants, chelating agents, antioxidants, stabilizers, emulsifying agents, suspending agents, gel-forming agents, ointment bases, penetration enhancers, perfumes, and skin protective agents.

It should be pointed out one object of the present invention is to provide a bioactive composition comprising the culture media having at least one of the ingredients listed in table 2.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogen phosphoric acid, diethylamine etc.

### Example 1

### Preparation of a Stock Solution

An alkoxy releasing copolymer suspension in water such as 30% (W/V) polymethylmethacrylate/polyethylmethacrylate (PMMA/PEMA) 30:70 was purchased commercially and used as a stock solution. The stock solution further comprises 0.15% surfactants such as sodium lauryl sulfate in weight out of the total weight of solids, and 1.2% methacrylic acid in weight out of the total weight of solids. The D[90] of the particles equals 1.99 micrometers. Similar suspensions having particle sizes between 1-5 microns were either used as monodisperse stock solutions and solutions comprising mixtures particle sizes thereof were also prepared. The zeta potential ranges between 30 to 130 mV at pH=7.0 depending on the ration of the particle sizes. The isoelectric point is kept by the adding a buffer solution comprising 50 ml 2M potassium phosphate and 29.1 ml 2M sodium hydroxide.

According to another embodiment of the present invention, a copolymer having ratio between said of ethylmethacrylate and said methylmethacrilate ranges from about 10:90 to about 90:10; the size of said polyalkylmethacrylate ranges from about 0.5 to about 5 microns; the viscosity of said polyalkylmethacrylate ranges from about 5 to about 28 cps units. There is about 31%-32% solids in said copolymer. According to said embodiment, the copolymer is diluted with a cell culture platform. The dilution is performed via 2 steps: the first one is dilution of 1:10 and the second step is a dilution of 1:100. It is optionally to add preservative to the copolymer.

### Example 2

### Preparation of a Biologically Active Composition (BAC)

A sample from the stock solution described above is diluted to a concentration of 0.025% and 1 part of it is admixed with 100 parts of a culture media. The culture media is admixed in a buffer solution comprising 50 ml 2M potassium phosphate and 29.1 ml 2M sodium hydroxide.

In a preferred embodiment the culture media comprises inter alia the following ingredients:

TABLE 1

| Ingredient | Quantity (mg per Liter) |
|---|---|
| calcium chloride dihydrate | 240-280 |
| sodium chloride | 5000-8000 |
| ferric nitrate nanohydrate | 0-0.30 |
| potassium chloride | 200-00 |
| magnesium sulfate | 94-99 |
| Sodium dihydrogen phosphate | 105-110 |
| L-alanine | 0-100 |
| L-arginine | 50-150 |
| L-cystine dihydrochloride | 50-100 |
| L-glutamine | 400-1000 |
| glycine | 20-50 |
| L-histidine | 10-60 |
| hydrochloride monohydrate | 0-20 |
| L-isoleucine | 90-120 |
| L-leucine | 50-200 |
| L-lysine hydrochloride | 125-200 |
| L-methionine | 10-70 |
| L-phenylalanine | 20-100 |
| L-serine | 30-100 |
| L-threonine | 10-150 |
| L-tryptophane | 0-50 |
| L-tyrosine disodium | 80-120 |
| L-valine | 80-110 |
| L-inositol | 5-30 |
| choline chloride | 1-5 |
| folic acid | 1-20 |
| nicotine amide | 1-15 |
| D-calcium pantothenate | 1-20 |
| riboflavin | 0.10-1.5 |
| thiamine hydrochloride | 1-10 |
| D-glucose | 1000-8000 |
| phenol red sodium | 2-20 |

In the most preferred embodiment the culture media contains inter alia the following ingredients:

TABLE 2

| Ingredient | Quantity (mg per Liter) |
|---|---|
| calcium chloride dihydrate | 264.90 |
| sodium chloride | 6400.00 |
| ferric nitrate nano + microhydrate | 0.10 |
| potassium chloride | 400.00 |
| magnesium sulfate | 97.66 |
| sodium dihydrogen phosphate | 108.69 |
| L-alanine | |
| L-arginine | 84.00 |
| L-cystine dihydrochloride | 62.58 |
| L-glutamine | 584.00 |
| glycine | 30.00 |
| L-histidine | 42.00 |
| Hydrochloride monohydrate | |
| L-isoleucine | 104.80 |
| L-leucine | 104.80 |
| L-lysine hydrochloride | 146.20 |
| L-methionine | 30.00 |
| L-phenylalanine | 66.00 |
| L-serine | 42.00 |
| L-threonine | 95.20 |
| L-tryptophane | 16.00 |
| L-tyrosine disodium | 103.79 |
| L-valine | 93.60 |
| L-inositol | 7.00 |
| choline chloride | 4.00 |
| folic acid | 4.00 |
| nicotine amide | 4.00 |
| D-calcium pantothenate | 4.00 |
| riboflavin | 0.40 |
| thiamine hydrochloride | 4.00 |
| D-glucose | 4500.00 |
| phenol red sodium | 15.34 |

It should be pointed out one object of the present invention is to provide a bioactive composition comprising the culture media having at least one of the ingredients listed in table 2.

### Example 3

### Preparation of a Biologically Active Solution (BAC)

A sample from a stock solution as described in example 1 was diluted in deionized water/ethyl alcohol 9:1 (V:V) to a concentration of 0.025% and 1 part of it is admixed with 100 parts of a culture media in a buffer.

### Example 4

### Preparation of a BAC

A sample from a stock solution as described in example 1 was diluted in deionized water/ethyl alcohol 19:1 (V:V) to a concentration of 0.04% and 1 part of it is admixed with 100 parts of a culture media in a buffer.

### Example 5

### Preparation of a BAC

A sample from a stock solution as described in example 1 was diluted in deionized water/ethyl alcohol 5:1 (V:V) to a concentration of 0.025% and 1 part of it is admixed with 100 parts of a culture media in a buffer.

### Example 6

### Preparation of a BAC

A sample from a stock solution as described in example 1 was diluted in deionized water/propanol 9:1(V:V) to a concentration of 0.09% and 1 part of it is admixed with 100 parts of a culture media in a buffer.

### Example 7

### Preparation of a BAC

A sample from a stock solution as described in example 1 was diluted in deionized water/propanol 19:1 (V:V) to a concentration of 0.025% and 1 part of it is admixed with 100 parts of a culture media in a buffer.

### Example 8

### Preparation of a BAC

A sample from a stock solution as described in example 1 was diluted in deionized water/propanol 5:1 (V:V) to a concentration of 0.05% and 1 part of it is admixed with 100 parts of a culture media in a buffer.

### Example 9

### Preparation of a BAC

A sample from a stock solution as described in example 1 was diluted in deionized water/iso-propanol 9:1 (V:V) to a concentration of 0.025% and 1 part of it is admixed with 100 parts of a culture media in a buffer.

### Example 10

### Preparation of a BAC

A sample from a stock solution as described in example 1 was diluted in deionized water/iso-propanol 19:1 (V:V) to a concentration of 0.025% and 1 part of it is admixed with 100 parts of a culture media in a buffer.

### Example 11

### Preparation of a BAC

A sample from a stock solution as described in example 1 was diluted in deionized water iso-propanol 5:1 (V:V) to a concentration of 0.055% and 1 part of it is admixed with 100 parts of a culture media in a buffer.

### Example 12

### Preparation of a BAC

A sample from a stock solution as described in example 1 was diluted in deionized water/iso-propanol/ethanol 9:0.5:0.5 (V:V) to a concentration of 0.025% and 1 part of it is admixed with 100 parts of a culture media in a buffer.

### Example 13

### Preparation of a BAC

A sample from a stock solution as described in example 1 was diluted in deionized water/iso-propanol/ethanol 19:0.2:0.8 (V:V) to a concentration of 0.025% and 1 part of it is admixed with 100 parts of a culture media in a buffer.

### Example 14

### Preparation of a BAC

A sample from a stock solution as described in example 1 was diluted in deionized water/iso-propanol/ethanol 6:1:1 (V:V) to a concentration of 0.025% and 1 part of it is admixed with 100 parts of a culture media in a buffer.

### Example 15

### Preparation of a BAC

A sample from a stock solution as described in example 1 was diluted in deionized water/iso-propanol/ethanol 6:1:1 (V:V) to a concentration of 0.05% and 1 part of it is admixed with 100 parts of a culture media in a buffer.

### Example 16

### Preparation of a BAC

A sample from a stock solution as described in example 1 was diluted in deionized water/iso-propanol/ethanol 6:1:1 (V:V) to a concentration of 0.01% and 1 part of it is admixed with 100 parts of a culture media in a buffer.

### Example 17

### Preparation of a BAC

A sample from a stock solution as described in example 1 was diluted in deionized water/iso-propanol/ethanol 6:1:1 (V:V) to a concentration of 0.09% and 1 part of it is admixed with 100 parts of a culture media in a buffer.

### Example 18

### Preparation of a BAC

A sample from a stock solution as described in example 1 was diluted in deionized water/iso-propanol/ethanol 6:1:1 (V:V) to a concentration of 0.09% and 2 part of it is admixed with 199 parts of a culture media in a buffer and 1 part of dichlorocarzol.

### Example 19

### Preparation of a BAC

A sample from a stock solution as described in example 1 was diluted in deionized water/iso-propanol/ethanol 20:1:1 (V:V) to a concentration of 0.09% and 2 part of it is admixed with 199 parts of a culture media in a buffer, 50 parts of a 10% solution of bovine serum albumin in deionized water and 1 part of dichlorocarzol.

### Example 20

### Preparation of a BAC

A sample from a stock solution as described in example 1 was diluted in deionized water/iso-propanol/ethanol 20:1:1 (V:V) to a concentration of 0.09% and 2 part of it is admixed with 199 parts of a culture media in a buffer, 50 parts of a 10% solution of bovine serum albumin in deionized water, 1 part of dichlorocarzol and 2 parts of 2% silver coated nano+microparticles in ethanol.

### Example 21

### Method for Treating a Wound

A chronic wound is pre-cleaned by a saline solution followed by application of 2 drops/cm² of the BAC to the wound until the wound is completely wet. After ten minutes an effective amount of chloramphenicol:Dermagran® 1:1 (w/w) is optionally added to the wound. The procedure is repeated between one to ten times a day, preferably one to three times a day, most preferably three times a day during a period of one week to four months, more preferably one to six weeks; possibly covering said wound with a protective dressing in between BAC applications.

### Example 22

### Method for Treating a Burn

A burn is pre-cleaned by a saline solution followed by application of one to five drops/cm², more preferably two drops/cm² of the BAC to the wound until the burn is completely wet. After ten minutes an effective amount of chloramphenicol:Dermagran® 1:1 (w/w) is optionally added to the wound. The procedure is repeated between one and ten times a day, preferably one and three times a day, most preferably three times a day during a period of one week to six months, more preferably one to six weeks; possibly covering the burn with a protective dressing in between BAC applications.

### Example 23

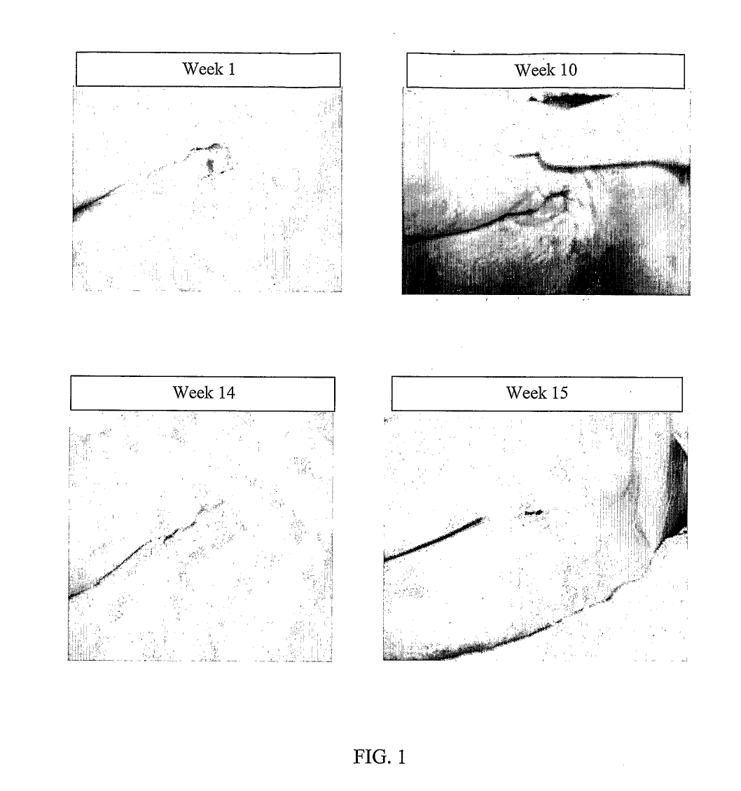

FIG. 1 shows the improvement in the condition of a 78 year old female patient, suffering from an incurable chronic pressure ulcer, which was treated irregularly between one and three times a day over a period of 16 week according to the method described in example 20.

### Example 24

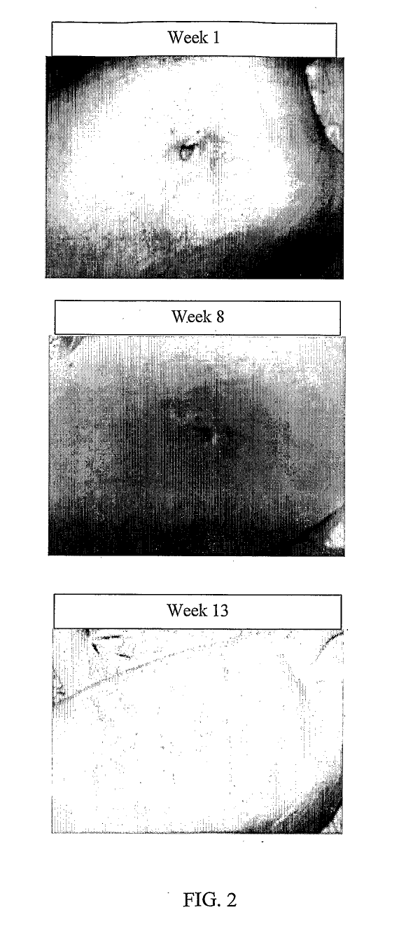

FIG. 2 shows the improvement in the condition of a 72 year old female patient, suffering from an incurable chronic pressure ulcer, which was treated irregularly between one and three times a day over a period of 16 week according to the method described in example 17.

### Example 25

### Dental and Orthopedics Use

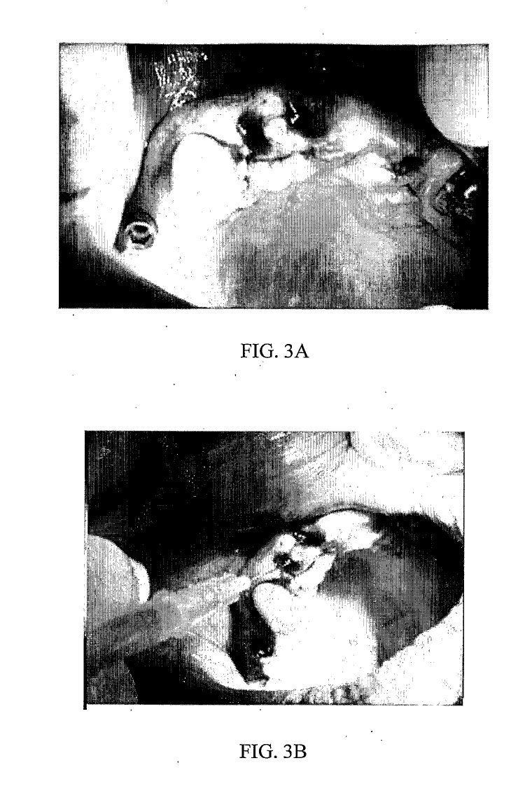

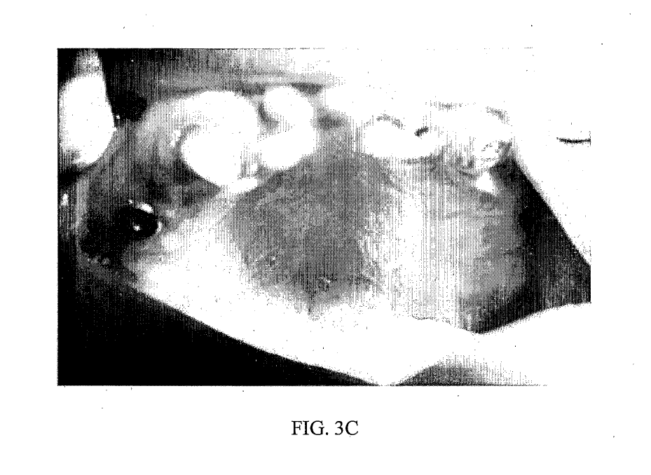

FIG. 3A illustrates the condition of a 62 years old male patient, administered regularly anti-coagulant drugs or blood thinning drugs. FIG. 3B illustrates the administration of the MPP and/or BAC as defined in the present invention. FIG. 3C illustrates the improvements in said patient's condition after merely 5 days.

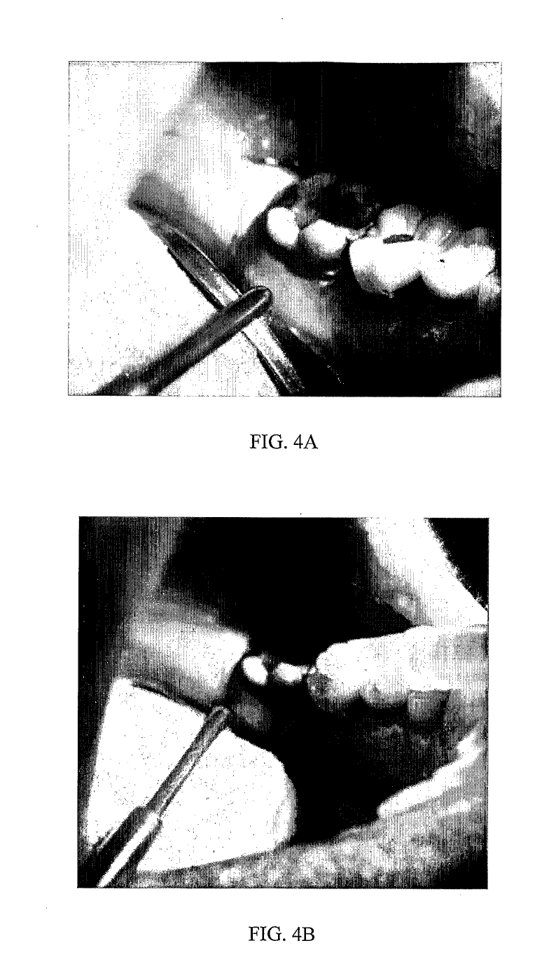

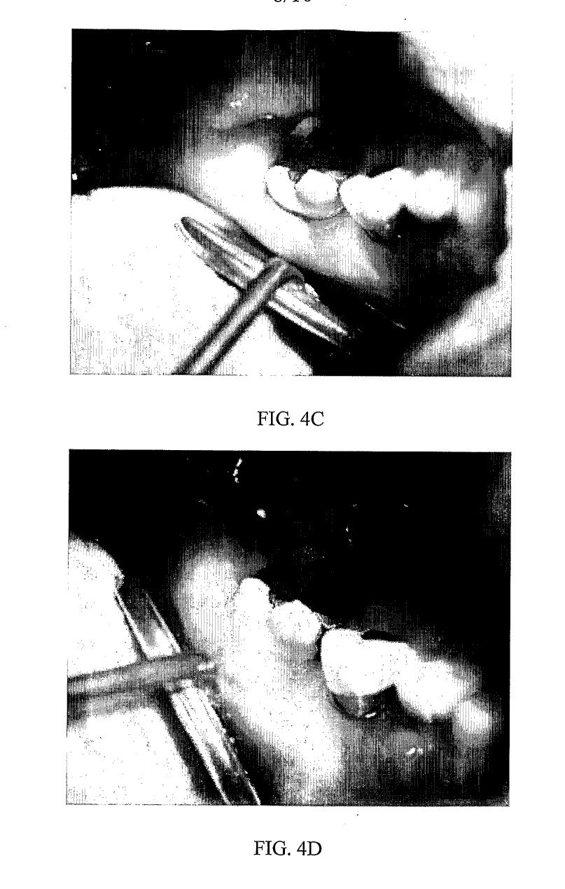

FIG. 4A illustrates the condition of a 52 years old female patient, administered regularly Coumadin and suffers from spontaneous bleeding. FIG. 4B illustrates the administration of the MPP and/or BAC as defined in the present invention. FIG. 4C illustrates the improvement in said patient's condition immediately after taking the MPP and/or BAC.

FIG. 4D illustrates the improvement in said patient's condition after merely 7 days. It should be emphasized that the MPP and/or BAC as defined in the present invention may be used for Endodontics, root canal treatment, Endodontics or any root diseases. Furthermore, the MPP and/or BAC may be adapted to be used as coagulator and as tissue fusion after tooth extraction. Yet more, the MPP and/or BAC as defined in the present invention can be used for Periodontitis, Periodontal Gum inflammation, Oral and maxillofacial surgery or in dental implants and bone construction.

Still more, the MPP and/or BAC as defined in the present invention can be used as mouthwash for oral diseases.

Still more, the MPP and/or BAC as defined in the present invention can be used in orthopedics in bone and soft tissues reconstructions, fusion treatment of bone fractures or fragment, different knee operations.

### Example 26

### Raynaud's Phenomenon

According to another embodiment of the present invention, the MPP and/or BAC as defined in the present invention can be used for treating Raynaud's Phenomenon.

A 50 years old female patient, who had Raynaud's Syndrome and was recommended to go through finger amputation, was administered with the MPP and/or BAC and her condition had dramatically improved.

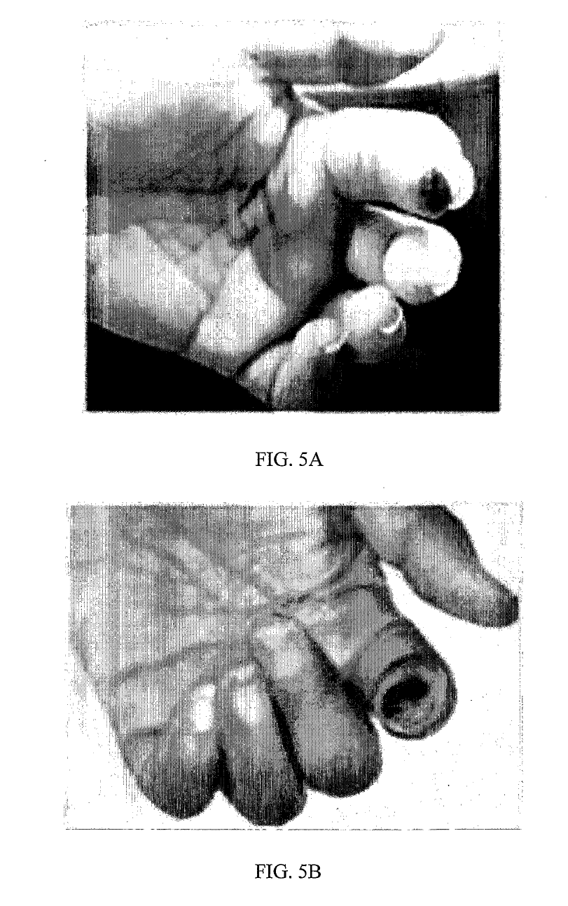

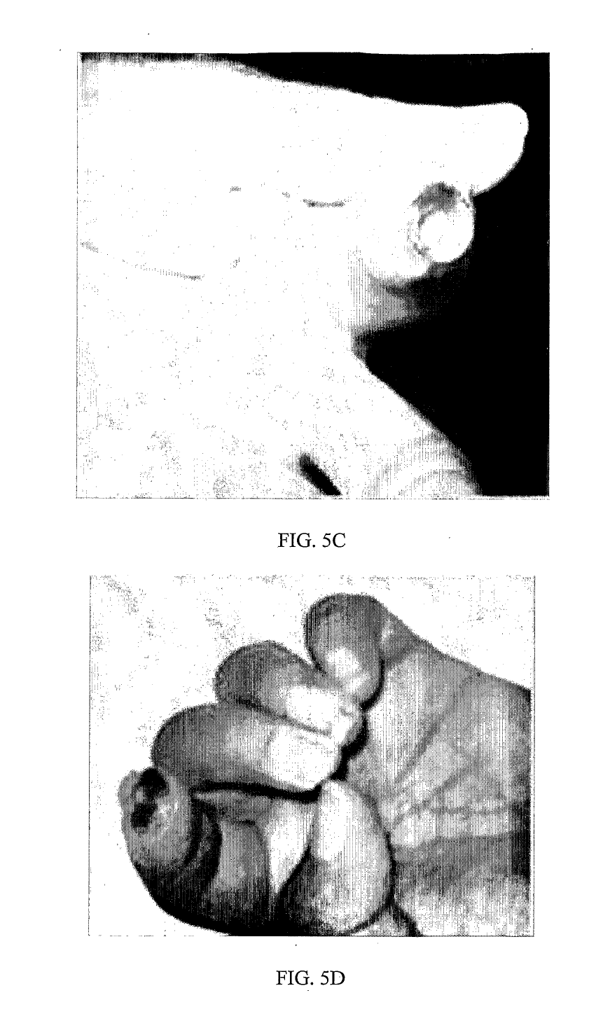

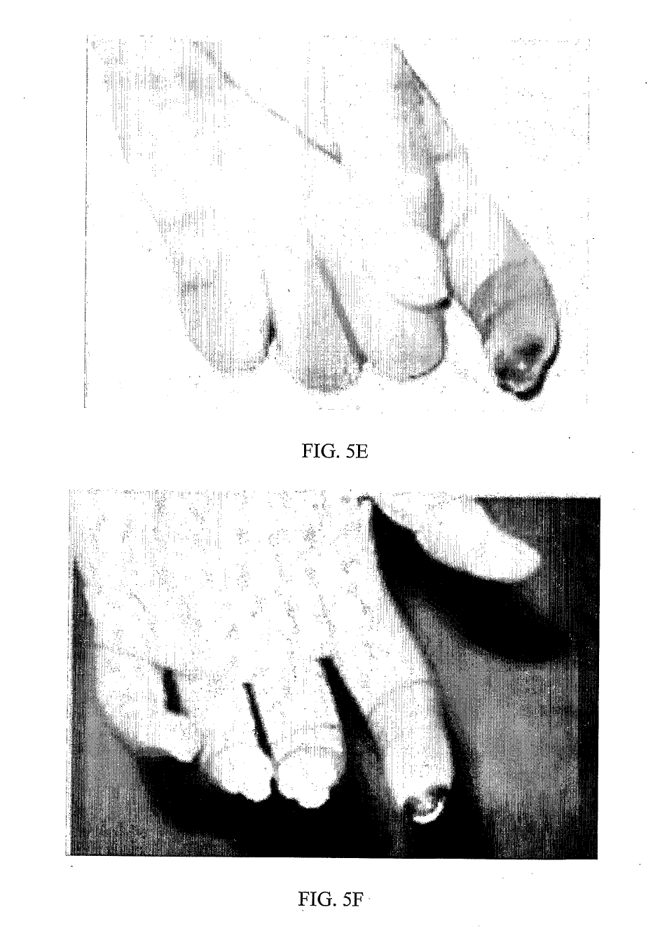

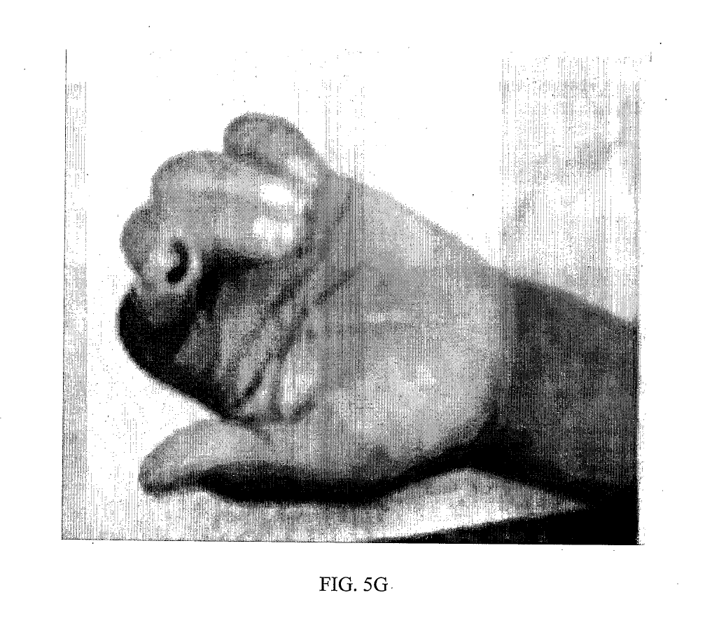

Reference is now made to FIGS. 5A-5G illustrating the improvements in said patient's condition after 1 day (see FIG. 5A), 2 days (see FIG. 5B), 3 days (see FIG. 5c), 4 days (see FIG. 5D), 6 days (see FIG. 5E), 13 days (see FIG. 5F) and 21 days (see FIG. 5G).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### REFERENCES

Vickery C. Exudate: what is it and what is its function?. In: Chemy G W, Harding K, editors. Management of wound exudate: Abstracts from the first combined meeting of the European Tissue Repair Society and the European Wound Management Association at Green College, University of Oxford. London: Churchill Communications, 1997.
Trengove N J, Bielefeldt-Ohmann H, Stacey M C. Mitogenic activity and cyokine levels in non-healing and healing chronic leg ulcers. Wound Rep Regen 2000; 8(1): 13-25.
Haeger K. The Illustrated History of Surgery. London: Harold Starke, 1989.
Cutting K F. Exudate: composition and functions. In: White R J, editor. Trends in Wound Care Volume III. London: Quay Books, 2004. 7. Wilson J W, Schurr M J, LeBlanc C L, Ramamurthy P, Buchanan K L, Nickerson C A. Mechanisms of bacterial pathogenicity. Postgrad Med J 2002; 78: 216-24.
Gautam N, Olofsson A M, Herwald H, Iversen L F, Lundgren-Akerlund E, Hedqvist P, et al. Heparin-binding protein (HBP/CAP37): a missing link in neutrophil-evoked alteration of vascular permeability. Nat Med 2001; 7(10): 1123-7.
Lundqvist K, Herwald H, Sonesson A, Schmidtchen A. Heparin binding protein is increased in chronic leg ulcer fluid and released from granulocytes by secreted products of Pseudomonas aeruginosa. Thromb Haemost 2004; 92(2): 281-7.
Kim S H, An H, Field K G, Wei C I, Velazquez J B, Ben-Gigirey B, et al. Detection of Morganella morganii, a prolific histamine former, by the polymerase chain reaction assay with 16S rDNA-targeted primers. J Food Prot 2003; 66(8): 1385-92.

1-38. (canceled)

39. Monodisperse Polymer Particles (MPP) adapted to release alkoxy groups by means of a hydrolyser, such that Monodisperse Bioactive Polymer Particles (MBPP) are obtained in vivo, said MBPP are characterized by
a. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
b. a long dimension between 0.1 and 10 micrometers; and
c. a zeta potential value of 30 to 130 mV at pH of about 7.0,
wherein said MBPP alter, inhibit, activate, induce or otherwise affect biological or chemical events in vivo.

40. The MPP according to claim 39, wherein the monomer of said MPP is said MBPP.

41. The MPP according to claim 39, wherein said alkoxy groups are characterized by a chemical formula of RO— in which R represents a radical having between 1 to 20 carbon atoms selected from the group consisting of aryl radicals, linear alkyl radicals, branched alkyl radicals, alkyl radicals comprising heteroatoms, unsaturated alkyl radicals, halogenated alkyl radicals, alkyl radical comprising cyclic residues, alkyl radical comprising heterocyclic residues, alkyl radicals comprising at least partially fused cyclic residues, alkyl radicals comprising at least partially fused heterocyclic residues, methoxy or any combination thereof.

42. The MPP according to claim 39, wherein said hydrolyser is selected from a group consisting of a combination of a proton and a water molecule, a combination of a Louis acid and a water molecule, a hydroxide, and an ester.

43. The MPP according to claim 39, wherein said MPP are adapted to release alkoxy groups by means of a hydrolysis reaction, said hydrolysis reaction is selected from a group consisting of hydrolysis of an ester, hydrolysis of an acyl halide, hydrolysis of an amide, hydrolysis of an ether and hydrolysis of an anhydride.

44. The MPP according to claim 39, wherein said polymer is selected from the group consisting of a polyalkylmethacrylate with 1 to 20 carbon atoms in the carbon chain of the alkyl residue which is potentially unsaturated, potentially branched and potentially halogenated, polymethacrylate, melamine, carboxylated melamine resin with 1 to 30 carbon atoms in the carbon chain of the carboxylate residue which is potentially unsaturated, potentially branched and potentially halogenated, magnetic carboxylated melamine resin with 1 to 30 carbon atoms in the carbon chain of the carboxylate residue which is potentially unsaturated, potentially branched and potentially halogenated, silicone, polyvinylcarboxylate with 1 to 30 carbon atoms in the carbon chain of the carboxylate residue which is potentially unsaturated potentially branched and potentially halogenated, polyvinylether with 1 to 30 carbon atoms in the carbon chain of the alkyl residue which is potentially unsaturated, potentially branched and potentially halogenated, derivatives and copolymers thereof.

45. The MPP according to claim 44, wherein at least one is being held true (a) said polyalkylmethacrylate is polymethylmethacrylate (PMMA); (b) said polyalkylmethacrylate is added with ethylmethacrylate and/or methylmethacrilate such that the ratio between said of ethylmethacrylate and said methylmethacrilate ranges from about 10:90 to 90:10; (c) the size of said polyalkylmethacrylate ranges from about 0.5 to about 5 microns; (d) the viscosity of said polyalkylmethacrylate ranges from about 5 to about 28 cps units; (e) narrow size distribution around 1 micrometer; or any combination thereof.

46. The MPP according to claim 39, wherein said in vivo is selected from the group consisting of a cut, an incision, an abrasion, a laceration, a fracture, a contusion, a burn, an amputation, and a joint causing a rheumatic pain in a mammal.

47. The MPP according to claim 39, wherein said BP is capable of functioning as an agent from the group consisting of a drug, especially anti-inflammatory agent, a coagulant, a hemostasis stimulant, a wound healing promoter, a proliferation stimulant, a tissue functional stimulant, a chelator, an anti-oxidant, an inducer, an inhibitor, a labeling agent and a mixture thereof.

48. The MPP according to claim 39, wherein said polymer forms particles wherein the external diameter of said particles ranges from about 0.1 to about 10 micrometers.

50. The MPP according to claim 39, said polymer further consisting of adsorbed biologically active ingredients selected from the group consisting of a drug, a nutrient, a coenzyme, a vitamin, a pro-vitamin, a coagulant, a hemostasis stimulant, a tissue an anti-oxidant, an inducer, an inhibitor, a labeling agent and a mixture thereof, wherein said adsorbed biologically active ingredients a slowly released from said polymer.

51. A biologically active composition (BAC) comprising
a. MPP adapted to release alkoxy groups such that MBPP characterized by
i. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
ii. a long dimension between 0.1 and 10 micrometers; and
iii. a zeta potential value of 30 to 130 mV at pH of about 7.0 are obtained;
b. a culture media, comprising ingredients that allow eukaryotic cell proliferation;
c. bovine serum albumin; and
d. an acceptable topical carrier.

52. The BAC according to claim 51, wherein the monomer of said MPP is said MBPP.

53. The BAC according to claim 51, further comprising metal coated polymer nano and microparticles; further wherein said metal is selected from the list consisting of tin, germanium, zinc, cadmium and silver.

54. The BAC according to claim 51, wherein the concentration of said polymer in said composition is between about 0.005 to about 10% (W/W).

55. The BAC according to claim 51, wherein said BAC formulated as a phase selected from the group consisting of gel, ointment, cream, liquid aerosol spray, and powder.

56. The BAC according to claim 51, wherein said BAC is embedded in a pad.

57. The BAC according to claim 51, wherein said composition is encapsulated within a biodegradable capsule.

58. The BAC according to claim 51, wherein said polymer is adapted to adsorb toxic metabolites from dead cells in the process of wound healing.

59. The BAC according to claim 51, wherein said biologically active composition is adapted to be at least one selected from a group consisting of (a) topically applied on a wound; (b) administrated by an administrating means; said administrating means selected from the group consisting of injection, oral ingestion, and oral application, nasal application, vaginal application, ear application, eye application, topical application, and anal application, or any combination thereof.

60. The BAC according to claim 51, further comprising a preservative; further wherein said preservative is selected from the group consisting of dichloro carbazole, methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol and mixtures thereof.

61. A method for treating a subject having a wound comprising steps of
a. obtaining a BAC comprising:
i. MPP adapted to release alkoxy groups such that MBPP characterized by
a. at least one naturally occurring or synthetic long molecular chain consisting of biologically stable backbones optionally crosslinked, further characterized by a molecular weight of at least 1 KD, comprising between 10 to 1,000,000 repeated covalently-linked small molecules with a functionality of at least one alkoxy releasing group per molecule;
b. a long dimension between 0.1 and 10 micrometers; and
c. a zeta potential value of 30 to 130 mV at pH of about 7.0 are obtained;
ii. a culture media, comprising ingredients that allow eukaryotic cell proliferation;
iii. bovine serum albumin; and
iv. an acceptable topical carrier;
b. cleaning said wound;
c. applying one to five drops/cm2 of said BAC to said wound until said burn is completely wet;
d. optionally applying an effective amount of chloramphenicol:Dermagran™ 1:1 (w/w) 10 minutes after step c;
e. possibly covering said wound with a protective dressing; and
f. repeating steps (b) through (e) at least once a day for a period of at least one week, until said wound is healed.

62. The method according to claim 61, wherein cleaning of the wound is done with a saline solution.

63. The method according to claim 61, wherein said wound is a burn.

64. The BAC according to claim 51, especially adapted for treating symptoms selected from a group consisting of Endodontics, root canal treatment, as coagulator, Periodontitis, Periodontal Gum inflammation, Oral and maxillofacial surgery or in dental implants and bone construction, orthopedics treatment in bone and soft tissues reconstructions, fusion treatment of bone fractures or fragment, different knee operations, Raynaud's Phenomenon or any combination thereof.

65. The BAC according to claim 51, formulated as mouthwash for oral diseases.
